SEQUENCE LISTING (61 sequences)

SEQ ID NO: 1 - DNA, 245 bp, Artificial Sequence
  Oligonucleotide primer for detection of Botrytis Oligonucleotide primer for detection of BotrytisBC_target1_41-45_hits_per_genome
aggaaaggatagtgtgtgaacggagtgaataacttcaattcaattaccactgtaatatagcaactataataaagccctaagcgaatgcgaaagagagtagctctttctgtaagcctttataaggcttactactttcgatacgtagctagctctttagacagaatacaattagacatacaggacctacgatattcgtgggtgctacgtcttccgtatccttctcgtaccaacagatagtgaggttg

SEQ ID NO: 2 - DNA, 179 bp, Artificial Sequence
  Oligonucleotide primer for detection of Botrytis Oligonucleotide primer for detection of BotrytisBC_target7_20-25_hits_per_genome
tgttacgacggattagtaacaggctgtagaatcaccaacgtataggctataatggtattataggcctcagtgattcagctgcagtataccgggggatactaggcatccaaggaaagccttaggtatatatatagtattaattatagaatattctaaaagtataggatacagtttttaga

SEQ ID NO: 3 - DNA, 133 bp, Artificial Sequence
  Oligonucleotide primer for detection of Botrytis Oligonucleotide primer for detection of BotrytisBC_target3_38-40_hits_per_genome_5S_ribosomal_RNA
tctgacacatacgaccatagactgaagagaattgggcatcccgtccgctctgccatacacaagcttcagatcggtggattagtagttgggtgggtgaccaccagcgaatccccactgttgtatgtttcttttc

SEQ ID NO: 4 - DNA, 194 bp, Artificial Sequence
  Oligonucleotide primer for detection of Botrytis Oligonucleotide primer for detection of BotrytisBC_target4_20-21_hits_per_genome
aatttagaactgttggtttcaccatggggatggtgaattcaatatagtactatggttcacactgttgtaatattgcttaaggttctaaaagctaagactacgaaacgtattgctgtagtgccgaaaggcgctagcacaagcgctagcacggtcacatgatcactatcccgacaagaaccatcactgtcctcaca

SEQ ID NO: 5 - DNA, 478 bp, Artificial Sequence
  Oligonucleotide primer for detection of Botrytis Oligonucleotide primer for detection of BotrytisBC_target5_4-20_hits_per_genome
taagttgagtaccccactttcggaccacccctcttttggaccacctaaaaatatatatatatatgaattttaaacttcaataactcaaccactattcaacttcaatacaatcccctgtagtgtcagtttcataaatactatcagagtcttttatctcaatttcccgatcaccagcttcaatttgagcttgatatatagcttctatccctgcaaacctcgaatttggactagtttttaccatccttctttttttaggtataatctcttctaatttttgctccaattgctttattcgtttcttggactgtacaagttcatagtccttagcatcaaatcctttttgaatctttcgaaaaagcagccggcgagttggaatatcggtctcatcaactttctccattatatcagcatattttcgaatatcacttcctttttgaggggttttccatgcaataaaagatgaatgtatttcctccat

SEQ ID NO: 6 - DNA, 593 bp, Artificial Sequence
  Oligonucleotide primer for detection of Botrytis Oligonucleotide primer for detection of BotrytisBC_target6_5-24_hits_per_genome
taagttgagtaccccactttcggaccacccctcttttggaccacctaaaatcttaccccattttaagcactacctcccaacttcatctttaataaatcaacaaccacatattcaattgatataagattttaatatattatcaattaagctataacaaagccttatactgaagataatattgctgcagcactttttgcaattgcagaaggcatgtctatatataaggcttactcagaatatgatattccccacaccactttatacaactatataaatagccacctttcacataaaaaagatacacaaaacctatagaagatagctcctatataggagagagctttagcaaattggattttaatacagaaagccctaaaaactagccctatctattatcaaatacaaaaattaggaaagtccattctcaacctcgaaagagatgatttatttttgaacaagcgatggatatatagttttttgaaaagaaacctagaaattaaaactaaaaggcaatataaaatcaataatgcctatatcaataatacaattaccaaaattataagcaagttctttgaaaaattagatttactaac

SEQ ID NO: 7 - DNA, 175 bp, Artificial Sequence
  Oligonucleotide primer for detection of Botrytis Oligonucleotide primer for detection of BotrytisBC_target8_8-18_hits_per_genome
agggttggcttgtgtcacggcgccaactacatgttctgtagttgccttcgtgccttaggcacggactactagcgtgccctgcttcctataagtagggcctcactcttccatagctcttccacccttatggtacaatatacgtctttacccgtgccttgacagtttgtaccatctt

SEQ ID NO: 8 - DNA, 304 bp, Artificial Sequence
  Oligonucleotide primer for detection of Botrytis Oligonucleotide primer for detection of BotrytisBC_target9_13-18_hits_per_genome
taataatttaatcttcttattgtaaaagagtagaaggtggtaatggtcacacaagaaaagccttcgcatatatcaagcatagagcaagtggctacacgtagtaaaatggggtgaatcactatattgcgatagcgaggtgagggaggcggtaagagctgggcactcaattcttcaggagacaactttagaaggtagaaaattcgatgatatttttaggtctacagaagtgaagctataaatactaaatgttgataacacgtgatcccagagtcacgtgttttcactctcacattatcgattggaa

SEQ ID NO: 9 - DNA, 369 bp, Artificial Sequence
  Oligonucleotide primer for detection of BotrytisOligonucleotide primer for detection of BotrytisBC_target10_26-27_hits_per_genome
atcactcacttacttcactttcaatttattcttcaatcagaagctttaccactataccatgccatacagattgtactatttatatacattatctacctagctttgatttatatattcatattcaattcaattactaatcgaattcaatataaataaagtattcatcatcttaaactagaattcaagaatttcactaagtccctttggaataaaaatttctaatcatttacaaaagaaaaagccccctaatcaatactttaaaaacgcttttttcaatacattataaaaatattgaatattttctggggctgataaagcggccacttcgtcaatcgcggattctgccaccacagggggtatatatactac

SEQ ID NO: 10 - DNA, 172 bp, Artificial Sequence
  Oligonucleotide primer for detection of Botrytis Oligonucleotide primer for detection of BotrytisBC_target11_8-14_hits_per_genome
ggtttatcagatcagtgagtgggtcatatcagtgaatgggtcatttgaaaatacatcaaaattaggcctttttcaatattcatattttaatagctaatcattattttgaaaaaagaaaagaattttcaataacaataagaaattagcttcttataaatttagttttttttat

SEQ ID NO: 11 - DNA, 300 bp, Artificial Sequence
  Oligonucleotide primer for detection of Botrytis Oligonucleotide primer for detection of BotrytisBC_IGS_1
tggttcgactgtagtccctaggaacgccctctgagtgtcctaggaatgcccccggtgagcccttggtctaaagccgtataggtgactagttaaccccatatagtttgtgcgagtacacacactactaccggtgagcaggctgtaatttcaatgtgcagaatctgtccccggtgagcgcaggtcaccttgcaatgagtggacagcatgtttgaaatgcgattaattgttgctcccggtgagcccactaaataattctgggagttggccatctcatatttcatccccggtgagcccaagata

SEQ ID NO: 12 - DNA, 928 bp, Artificial Sequence
  Oligonucleotide primer for detection of Botrytis Oligonucleotide primer for detection of BotrytisBC_tubulin_gene
acatcagatatctattcctcgccctcaattgggacctcctcttcgtactcctcctctccctcagagatcgaggcatcctggtattgttgatactcggaaaccaaatcgttcatgttggactcagcctcagtgaactccatctcgtccataccttcaccagtgtaccaatgcaagaaagcctttcttctgaacatagcagtgaattgatcaccgacacgcttgaaaagttcttggatggatgtcgagttaccaacgaaggtggaggacatcttgagaccacggggaggaatggagcaaagggcggtttggacgttgttagggatccactcaacgaagtaggatgagttcttgttttggacgttgcgcatttggtcctcaacctccttcatggaaaccttaccacggctacagaaagttagtttctacaagattttggcagattgattacagggcaaacttacaaaatggcagagcatgtcaagtaacgaccgttacggaaatcggaagcggccatcatgttcttagggtcgtacatttgttgagtcaactctggaacggtgacagcacggaaagagtgtgcgccacgactggtcaaaggagcaaatccaaccatgaagaaatggagacgggggaatggaaccatgttaacagccaactttcggagatctgagttaagttgaccagggaaacggagacaggtggtaacaccggacatgacggcggaaaccaagtggttaagatctccgtaagatgggttgctgagcttcaaggttctcatgcaaatatcgtaaagagcctcgttatcgatacagaaggtcgcgtcagagttctcaaccaattgatggacagagagagttgcgttatatggctcgacaacggtatcggaaacctttggcgatgggacgacggagaaggtagccatcatacg

SEQ ID NO: 13 - DNA, 921 bp, Artificial Sequence
  Oligonucleotide primer for detection of Botrytis Oligonucleotide primer for detection of BotrytisBC_Cutinase
aaaagaatctcaacttaaatggaaattcattctgagctgatactcgttgccgtcacataaaatataaagtgattgacatcgagaaagtttctcaatctacctagtttgcatcgctttgagcaactcatcactccggctcggcagatgttagctcgaatgaaagatttgatggtaggctttcctgtcgaatttgccagttgaatttgccagtatggtgtgaatgcgctgtatgttctagcgacgcctaatactagatgtctaagatgtctagtagtagctcgacgccgtgacatgccgtcaccatgaaatttgctgagtttggttgtataaagaagagggaaaggaatgaaaaccaatacacggagagaaatagtataaagattggattgaatggaaagtgtttacttcctctgcgttaactctagtttccggatagtaccgcgggatcttgctgggcaggcatgagctatgtggagcttcaagctttctcaatatggggtagccttatgtcccttcccttgtccttgctgtcgatctcaccattttccatttctcttcacctctttctcctccgtgattcaaccacacctcttagaatctttaatgcctcggcagttgaagacatacacgggcctcgtcaattatcgcacattgtactactcaccaacttaatgaaatactggcatctaaacacggtattcaaaagatgcgagatgtacagacagacactcgcaggtcatgacaaattccccgtcggacttccacattggaattttgagagtccaagcaaaaaagttacaatggtgttatgttgcatcacaatcaaatcttccttactttttctccacacagccaccaccatcctccttatgcttctttcatccttaacgtttcaaaaagtcggattcatctgaaaaagtt

SEQ ID NO: 14 - DNA, 20 bp, Artificial Sequence
  Oligonucleotide primer for detection of Botrytis Oligonucleotide primer for detection of BotrytisBC_target1_F1
cctaagcgaatgcgaaagag

SEQ ID NO: 15 - DNA, 20 bp, Artificial Sequence
  Oligonucleotide primer for detection of Botrytis Oligonucleotide primer for detection of BotrytisBC_target1_R1
cgagaaggatacggaagacg

SEQ ID NO: 16 - DNA, 21 bp, Artificial Sequence
  Oligonucleotide primer for detection of Botrytis Oligonucleotide primer for detection of BotrytisBC_target7_F1
caggctgtagaatcaccaacg

SEQ ID NO: 17 - DNA, 20 bp, Artificial Sequence
  Oligonucleotide primer for detection of Botrytis Oligonucleotide primer for detection of BotrytisBC_target7_R1
ctaaggctttccttggatgc

SEQ ID NO: 18 - DNA, 20 bp, Artificial Sequence
  Oligonucleotide primer for detection of Botrytis Oligonucleotide primer for detection of BotrytisBC_target3_F1
ctgaagagaattgggcatcc

SEQ ID NO: 19 - DNA, 20 bp, Artificial Sequence
  Oligonucleotide primer for detection of Botrytis Oligonucleotide primer for detection of BotrytisBC_target3_R1
catacaacagtggggattcg

SEQ ID NO: 20 - DNA, 19 bp, Artificial Sequence
  Oligonucleotide primer for detection of Botrytis Oligonucleotide primer for detection of BotrytisBC_target4_F1
caccatggggatggtgaat

SEQ ID NO: 21 - DNA, 20 bp, Artificial Sequence
  Oligonucleotide primer for detection of Botrytis Oligonucleotide primer for detection of BotrytisBC_target4_R1
ttcggcactacagcaatacg

SEQ ID NO: 22 - DNA, 20 bp, Artificial Sequence
  Oligonucleotide primer for detection of Botrytis Oligonucleotide primer for detection of BotrytisBC_target5_F1
ccctcttttggaccacctaa

SEQ ID NO: 23 - DNA, 20 bp, Artificial Sequence
  Oligonucleotide primer for detection of Botrytis Oligonucleotide primer for detection of BotrytisBC_target5_R1
ctggtgatcgggaaattgag

SEQ ID NO: 24 - DNA, 21 bp, Artificial Sequence
  Oligonucleotide primer for detection of Botrytis Oligonucleotide primer for detection of BotrytisBC_target6_F1
aagcactacctcccaacttca

SEQ ID NO: 25 - DNA, 19 bp, Artificial Sequence
  Oligonucleotide primer for detection of Botrytis Oligonucleotide primer for detection of BotrytisBC_target6_R1
gcaattgcaaaaagtgctg

SEQ ID NO: 26 - DNA, 20 bp, Artificial Sequence
  Oligonucleotide primer for detection of Botrytis Oligonucleotide primer for detection of BotrytisBC_target8_F1
ctactagcgtgccctgcttc

SEQ ID NO: 27 - DNA, 20 bp, Artificial Sequence
  Oligonucleotide primer for detection of Botrytis Oligonucleotide primer for detection of BotrytisBC_target8_R1
aaggcacgggtaaagacgta

SEQ ID NO: 28 - DNA, 21 bp, Artificial Sequence
  Oligonucleotide primer for detection of Botrytis Oligonucleotide primer for detection of BotrytisBC_target9_F1
catagagcaagtggctacacg

SEQ ID NO: 29 - DNA, 20 bp, Artificial Sequence
  Oligonucleotide primer for detection of Botrytis Oligonucleotide primer for detection of BotrytisBC_target9_R1
ttgagtgcccagctcttacc

SEQ ID NO: 30 - DNA, 35 bp, Artificial Sequence
  Oligonucleotide primer for detection of Botrytis Oligonucleotide primer for detection of BotrytisTARGET1_TDX_1F
aagccctaagcgaatgcgaaagagactagctcttt

SEQ ID NO: 31 - DNA, 35 bp, Artificial Sequence
  Oligonucleotide primer for detection of Botrytis Oligonucleotide primer for detection of BotrytisTARGET1_TDX_2F
tartaaagccctaagcgaatgcgaaagagactagc

SEQ ID NO: 32 - DNA, 35 bp, Artificial Sequence
  Oligonucleotide primer for detection of Botrytis Oligonucleotide primer for detection of BotrytisTARGET1_TDX_3F
wactgtartaaagccctaagcgaatgcgaaagaga

SEQ ID NO: 33 - DNA, 35 bp, Artificial Sequence
  Oligonucleotide primer for detection of Botrytis Oligonucleotide primer for detection of BotrytisTARGET1_TDX_4F
atagcwactgtartaaagccctaagcgaatgcgaa

SEQ ID NO: 34 - DNA, 35 bp, Artificial Sequence
  Oligonucleotide primer for detection of Botrytis Oligonucleotide primer for detection of BotrytisTARGET1_TDX_5F
gtaatatagcwactgtartaaagccctaagcgaat

SEQ ID NO: 35 - DNA, 35 bp, Artificial Sequence
  Oligonucleotide primer for detection of Botrytis Oligonucleotide primer for detection of BotrytisTARGET1_TDX_6F
ccactgtaatatagcwactgtartaaagccctaag

SEQ ID NO: 36 - DNA, 35 bp, Artificial Sequence
  Oligonucleotide primer for detection of Botrytis Oligonucleotide primer for detection of BotrytisTARGET1_TDX_7F
aattaccactgtaatatagcwactgtartaaagcc

SEQ ID NO: 37 - DNA, 36 bp, Artificial Sequence
  Oligonucleotide primer for detection of Botrytis Oligonucleotide primer for detection of BotrytisTARGET1_TDX_8F
aattcaattaccactgtaatatagcwactgtartaa

SEQ ID NO: 38 - DNA, 35 bp, Artificial Sequence
  Oligonucleotide primer for detection of Botrytis Oligonucleotide primer for detection of BotrytisTARGET1_TDX_1R
gttggtacgagaaggatacggaagacgtagcaccc

SEQ ID NO: 39 - DNA, 35 bp, Artificial Sequence
  Oligonucleotide primer for detection of Botrytis Oligonucleotide primer for detection of BotrytisTARGET1_TDX_2R
tacgagaaggatacggaagacgtagcacccacgaa

SEQ ID NO: 40 - DNA, 35 bp, Artificial Sequence
  Oligonucleotide primer for detection of Botrytis Oligonucleotide primer for detection of BotrytisTARGET1_TDX_3R
gaaggatacggaagacgtagcacccacgaatwkcg

SEQ ID NO: 41 - DNA, 35 bp, Artificial Sequence
  Oligonucleotide primer for detection of Botrytis Oligonucleotide primer for detection of BotrytisTARGET1_TDX_4R
atacggaagacgtagcacccacgaatwkcgtaggt

SEQ ID NO: 42 - DNA, 35 bp, Artificial Sequence
  Oligonucleotide primer for detection of Botrytis Oligonucleotide primer for detection of BotrytisTARGET1_TDX_5R
gaagacgtagcacccacgaatwkcgtaggtcctgt

SEQ ID NO: 43 - DNA, 35 bp, Artificial Sequence
  Oligonucleotide primer for detection of Botrytis Oligonucleotide primer for detection of BotrytisTARGET1_TDX_6R
cgtagcacccacgaatwkcgtaggtcctgtatgtc

SEQ ID NO: 44 - DNA, 35 bp, Artificial Sequence
  Oligonucleotide primer for detection of Botrytis Oligonucleotide primer for detection of BotrytisTARGET1_TDX_7R
cacccacgaatwkcgtaggtcctgtatgtctaatt

SEQ ID NO: 45 - DNA, 35 bp, Artificial Sequence
  Oligonucleotide primer for detection of Botrytis Oligonucleotide primer for detection of BotrytisTARGET1_TDX_8R
acgaatwkcgtaggtcctgtatgtctaattgtatt

SEQ ID NO: 46 - DNA, 35 bp, Artificial Sequence
  Oligonucleotide primer for detection of Botrytis Oligonucleotide primer for detection of BotrytisRibosomal_IGS_TDx_1F
cggtgagcaggctgtaatttcaatgtgcagaatct

SEQ ID NO: 47 - DNA, 35 bp, Artificial Sequence
  Oligonucleotide primer for detection of Botrytis Oligonucleotide primer for detection of BotrytisRibosomal_IGS_TDx_2F
actaccggtgagcaggctgtaatttcaatgtgcag

SEQ ID NO: 48 - DNA, 35 bp, Artificial Sequence
  Oligonucleotide primer for detection of Botrytis Oligonucleotide primer for detection of BotrytisRibosomal_IGS_TDx_3F
acactactaccggtgagcaggctgtaatttcaatg

SEQ ID NO: 49 - DNA, 35 bp, Artificial Sequence
  Oligonucleotide primer for detection of Botrytis Oligonucleotide primer for detection of BotrytisRibosomal_IGS_TDx_4F
tacacacactactaccggtgagcaggctgtaattt

SEQ ID NO: 50 - DNA, 35 bp, Artificial Sequence
  Oligonucleotide primer for detection of Botrytis Oligonucleotide primer for detection of BotrytisRibosomal_IGS_TDx_5F
gcgagtacacacactactaccggtgagcaggctgt

SEQ ID NO: 51 - DNA, 35 bp, Artificial Sequence
  Oligonucleotide primer for detection of Botrytis Oligonucleotide primer for detection of BotrytisRibosomal_IGS_TDx_6F
tttgtgcgagtacacacactactaccggtgagcag

SEQ ID NO: 52 - DNA, 35 bp, Artificial Sequence
  Oligonucleotide primer for detection of Botrytis Oligonucleotide primer for detection of BotrytisRibosomal_IGS_TDx_7F
tatagtttgtgcgagtacacacactactaccggtg

SEQ ID NO: 53 - DNA, 35 bp, Artificial Sequence
  Oligonucleotide primer for detection of Botrytis Oligonucleotide primer for detection of BotrytisRibosomal_IGS_TDx_8F
ccccatatagtttgtgcgagtacacacactactac

SEQ ID NO: 54 - DNA, 35 bp, Artificial Sequence
  Oligonucleotide primer for detection of Botrytis Oligonucleotide primer for detection of BotrytisRibosomal_IGS_TDx_1R
gtgggctcaccgggagcaacaattaatcgcatttc

SEQ ID NO: 55 - DNA, 35 bp, Artificial Sequence
  Oligonucleotide primer for detection of Botrytis Oligonucleotide primer for detection of BotrytisRibosomal_IGS_TDx_2R
atttagtgggctcaccgggagcaacaattaatcgc

SEQ ID NO: 56 - DNA, 35 bp, Artificial Sequence
  Oligonucleotide primer for detection of Botrytis Oligonucleotide primer for detection of BotrytisRibosomal_IGS_TDx_3R
gaattatttagtgggctcaccgggagcaacaatta

SEQ ID NO: 57 - DNA, 35 bp, Artificial Sequence
  Oligonucleotide primer for detection of Botrytis Oligonucleotide primer for detection of BotrytisRibosomal_IGS_TDx_4R
tcccagaattatttagtgggctcaccgggagcaac

SEQ ID NO: 58 - DNA, 35 bp, Artificial Sequence
  Oligonucleotide primer for detection of Botrytis Oligonucleotide primer for detection of BotrytisRibosomal_IGS_TDx_5R
ccaactcccagaattatttagtgggctcaccggga

SEQ ID NO: 59 - DNA, 35 bp, Artificial Sequence
  Oligonucleotide primer for detection of Botrytis Oligonucleotide primer for detection of BotrytisRibosomal_IGS_TDx_6R
gatggccaactcccagaattatttagtgggctcac

SEQ ID NO: 60 - DNA, 35 bp, Artificial Sequence
  Oligonucleotide primer for detection of Botrytis Oligonucleotide primer for detection of BotrytisRibosomal_IGS_TDx_7R
tatgagatggccaactcccagaattatttagtggg

SEQ ID NO: 61 - DNA, 35 bp, Artificial Sequence
  Oligonucleotide primer for detection of Botrytis Oligonucleotide primer for detection of BotrytisRibosomal_IGS_TDx_8R
tgaaatatgagatggccaactcccagaattattta

(19) United States
(12) Patent Application Publication
(10) Pub. No.: US 2016/0076110 A1
(43) Pub. Date: Mar. 17, 2016

(54) METHODS FOR PATHOGEN DETECTION AND DISEASE MANAGEMENT ON MEATS, PLANTS, OR PLANT PARTS

(71) Applicant: AGROFRESH INC., COLLEGEVILLE, PA (US)

(72) Inventors: William T. BEESON, IV, Indianapolis, IN (US); Daniel MACLEAN, Woodland, CA (US); Christina COEN, Zionsville, IN (US)

(21) Appl. No.: 14/850,287

(22) Filed: Sep. 10, 2015

Related U.S. Application Data
(60) Provisional application No. 62/049,080, filed on Sep. 11, 2014.

(51) Int. Cl. C12Q 1/68

(52) U.S. Cl. CPC C12Q 1/6895; C12Q 2600/16

(57) ABSTRACT

Provided are methods for detecting pathogens affecting meats, plants, or plant parts. Also provided are methods for predicting disease and/or disease management for meats, plants, or plant parts. In some embodiments, methods provided comprise nucleic acid based amplification. Examples of such nucleic acid based amplification methods include quantitative polymerase chain reaction (qPCR) and recombinase polymerase amplification (RPA).

### CROSS-REFERENCE TO RELATED APPLICATION

This application claims the benefit under 35 USC §119(e) of U.S. Provisional Application Ser. No. 62/049,080, filed on Sep. 11, 2014, the entire disclosure of which is incorporated herein by reference.

### BACKGROUND OF THE INVENTION

Various fruits after harvest can be subject to pathogens and disease development as a consequence. For examples, berries including strawberry are typically hand-pick upon harvest and are subject to mold and later become rotten as a consequence.

Thus, there remains a need to develop methods for detecting pathogens on meats, plants, or plant parts. In addition, detection of pathogens can enable better disease management for meats, plants, or plant parts if interest.

### SUMMARY OF THE INVENTION

Provided are methods for detecting pathogens affecting meats, plants, or plant parts. Also provided are methods for predicting disease and/or disease management for meats, plants, or plant parts. In some embodiments, methods provided comprise nucleic acid based amplification. Examples of such nucleic acid based amplification methods include quantitative polymerase chain reaction (qPCR) and recombinase polymerase amplification (RPA).

Specifically, provided are sequences of oligonucleotide primer sets for detection of Botrytis. In one embodiment, the primer sets provided can be used for RPA.

In addition, combinations of primers at varying sensitivities for Botrytis detection are provided for disease management for determining risk level of disease development related to Botrytis infection. For example, a three tier risk levels consisting of low risk, medium risk, and high risk can be provided.

Also provided are methods of sampling calyx of strawberries for Botrytis detection.

### BRIEF DESCRIPTION OF THE DRAWINGS

FIG. 1 shows representative results of initial RPA primer screen for the Botrytis cinerea Ribosomal IGS target. Melt curve analysis shows a strong amplification primer pair (R1F1) and good amplification at 10 copies of Botrytis cinerea genomic DNA.

FIG. 2A and FIG. 2B show representative photos of BioAnalyzer analysis using R1F1 primers (FIG. 2A) or R1F6 primers (FIG. 2B). For FIG. 2A, the desired amplicon is ˜120 base pairs. The negative control shows no background desired amplicon product. Strong amplification is observed at 5 Botrytis genomic DNA copies. No significant artifact amplification is observed at 10 Botrytis genomic DNA copies, showing the higher sensitivity of the R1F1 primer pair when compared to the R1F6 primer pair. For FIG. 2B, the desired amplicon is ˜148 base pairs. The negative control shows no background desired amplicon product. Strong amplification is observed at 200-500 Botrytis genomic DNA copies. No amplification is observed at 50 Botrytis genomic DNA copies.

FIG. 3 shows representative photos of BioAnalyzer analysis of the products of RPA reactions with R1F1 and R1F6. The reactions are performed with forward primer F1 or F6.

FIG. 4 shows fluorescence as a function of amplification time. In the absence of target DNA, there can still be an increase in fluorescence.

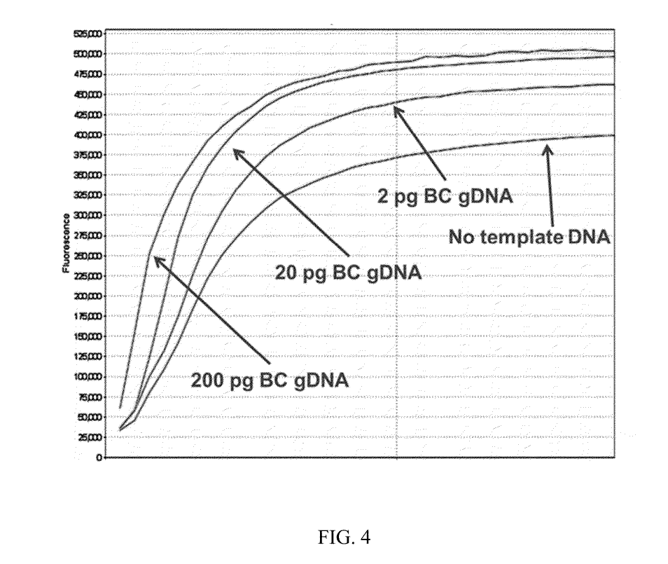

FIG. 5 shows electropherograms for the RPA and PCR reaction products of the R2F3 primer pair. Broad (PCR) or multiple products (RPA) are identified.

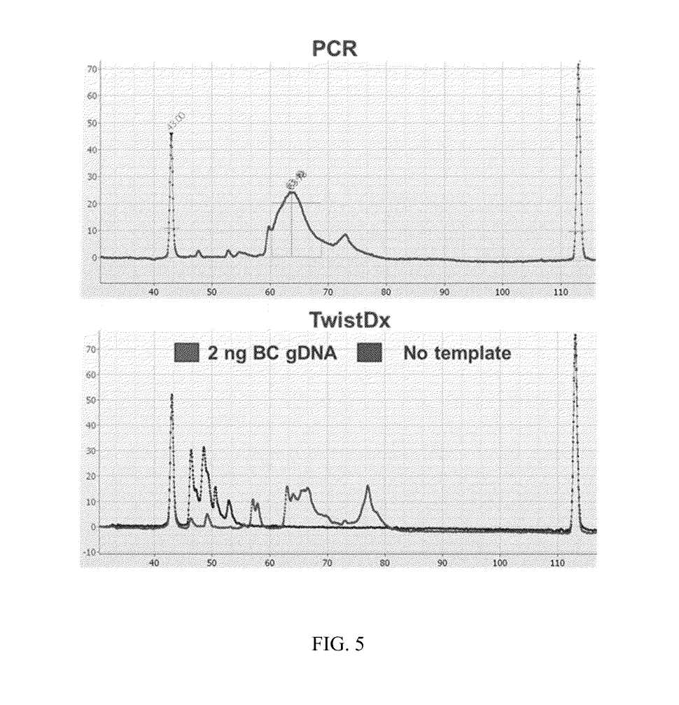

FIG. 6 shows electropherograms of the R1F1 and R1F3 RPA reaction amplicons. With no template present, multiple artifact amplicons are present. With 250 copies of Botrytis cinerea gDNA the desired amplicon is specifically produced.

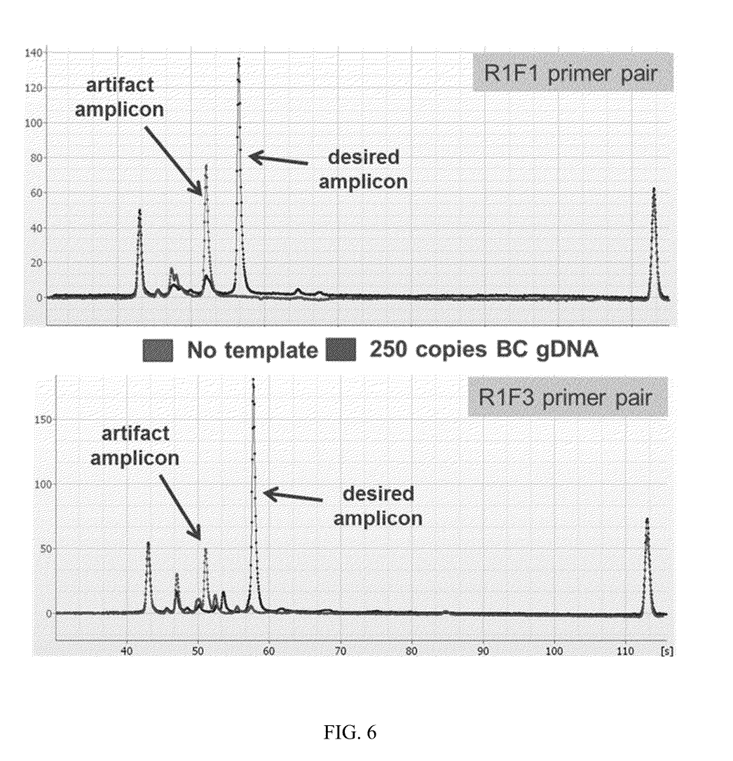

FIG. 7 shows analysis of RPA reaction products for proof-of-concept experiment. The negative sample only contained the RPA mastermix and primer pair. The calyx #1 and calyx #2 samples show the amplification products from two separately prepared calyxes. The calyx+BC samples are for calyx that is spiked with intact Botrytis spores. The BC only sample contained only Botrytis spores and no calyx material.

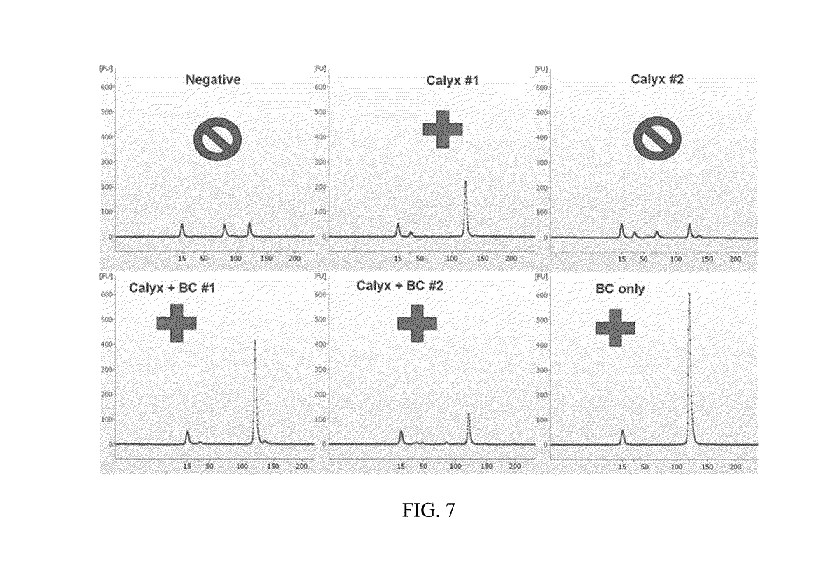

FIG. 8 shows primers ordered for development of RPA assay for Target 1. The primers listed in the lower panel were purchased as the reverse complement of the sequence shown.

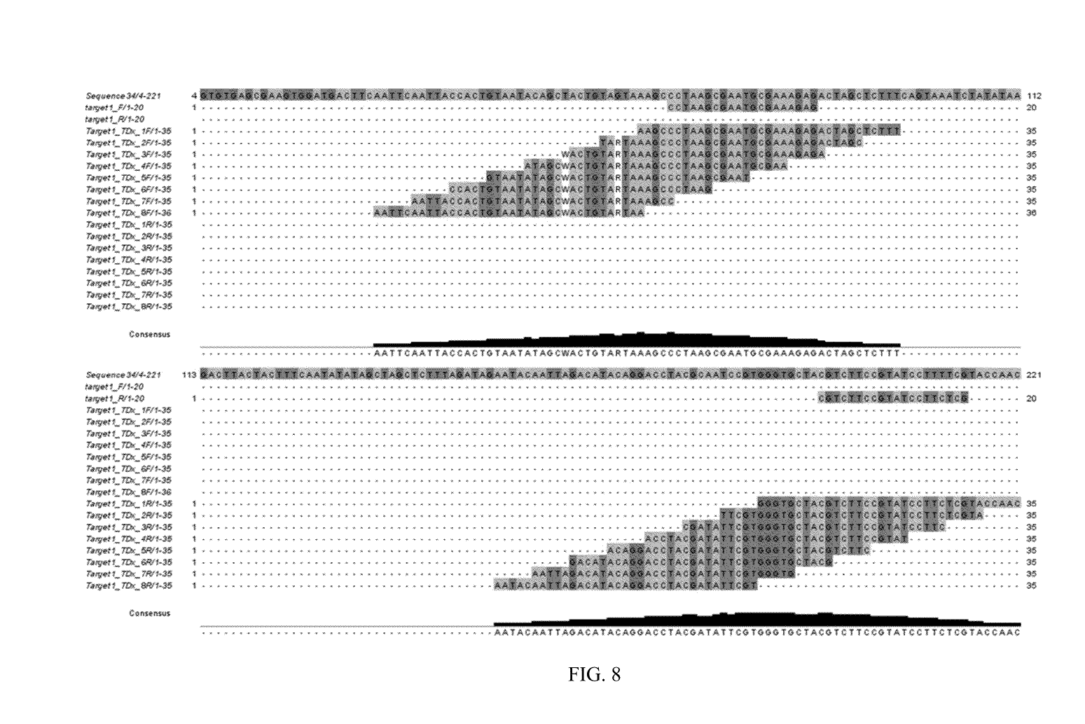

FIG. 9 shows ribosomal IGS primers ordered for RPA reactions.

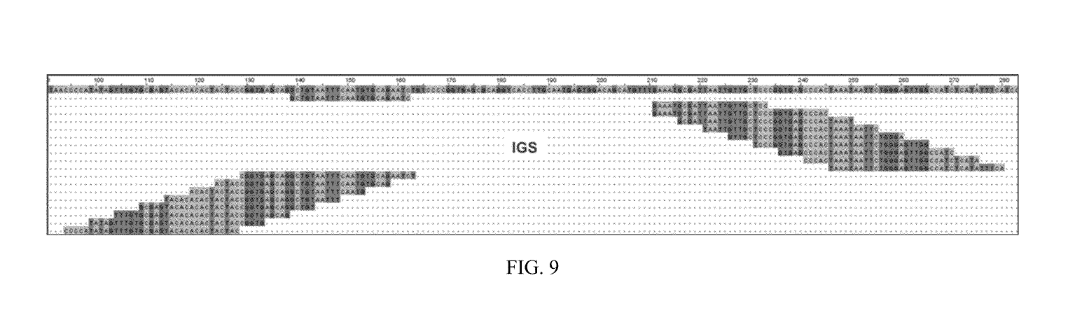

### DETAILED DESCRIPTION OF THE INVENTION

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise.

Diagnostic kits for detecting pathogens (for example Botrytis cinerea) are provided. Such diagnostic kits can be used by users/participants in the berry value chain (for example strawberry) to predict risk of Botrytis rot. In one embodiment, the diagnostic kits provided comprise an isothermal nucleic acid based test, for example recombinase polymerase amplification (RPA).

A risk model that correlates the amount of Botrytis on the sample with the probability of spoilage is also provided. In one embodiment, the diagnostic kits provided can detect from 10 to 10,000 spores; from 25 to 5,000 spores; from 50 to 2,500 spores; or from 100 to 1,000 spores of pathogen per strawberry calyx.

In one embodiment, the diagnostic kits provided enable detection of presence or absence of pathogens. In another embodiment, the diagnostic kits provided also enable quantitative and/or semi-quantitative (for example a multi-tier risk level system) detection of pathogens. In a further embodiment, the capability of quantitative and/or semi-quantitative detection is enabled by use/combinations of multiple primer sets of differing sensitivities for an isothermal nucleic acid based test, for example RPA.

The recombinase polymerase amplification (RPA) has been previously disclosed in U.S. Pat. Nos. 7,485,428, 7,666,598, and 7,763,427, the contents of which are thereby incorporated by reference in their entries.

In one aspect, provided are methods for detecting at least one pathogen affecting meats, plants, or plant parts. The methods comprise
(a) providing a sample of the meats, plants, or plant parts;
(b) performing a nucleic acid based amplification from the sample using a plurality of oligonucleotide primers for at least one target sequence; and
(c) determining presence or absence of the at least one pathogen from the sample.

In one embodiment of the methods provided, the nucleic acid based amplification comprises quantitative polymerase chain reaction (qPCR) or recombinase polymerase amplification (RPA). In another embodiment, the nucleic acid based amplification comprises isothermal nucleic acid amplification. In a further embodiment, the nucleic acid based amplification comprises recombinase polymerase amplification (RPA).

In another embodiment, the at least one pathogen is selected from the group consisting of Acremonium spp., Albugo spp., Alternaria spp., Ascochyta spp., Aspergillus spp., Botryodiplodia spp., Botryospheria spp., Botrytis spp., Byssochlamys spp., Candida spp., Cephalosporium spp., Ceratocystis spp., Cercospora spp., Chalara spp., Cladosporium spp., Colletotrichum spp., Cryptosporiopsis spp., Cylindrocarpon spp., Debaryomyces spp., Diaporthe spp., Didymella spp., Diplodia spp., Dothiorella spp., Elsinoe spp., Fusarium spp., Geotrichum spp., Gloeosporium spp., Glomerella spp., Helminthosporium spp., Khuskia spp., Lasiodiplodia spp., Macrophoma spp., Macrophomina spp., Microdochium spp., Monilinia spp., Monilochaethes spp., Mucor spp., Mycocentrospora spp., Mycosphaerella spp., Nectria spp., Neofabraea spp., Nigrospora spp., Penicillium spp., Peronophythora spp., Peronospora spp., Pestalotiopsis spp., Pezicula spp., Phacidiopycnis spp., Phoma spp., Phomopsis spp., Phyllosticta spp., Phytophthora spp., Polyscytalum spp., Pseudocercospora spp., Pyricularia spp., Pythium spp., Rhizoctonia spp., Rhizopus spp., Sclerotium spp., Sclerotinia spp., Septoria spp., Sphaceloma spp., Sphaeropsis spp., Stemphyllium spp., Stilbella spp., Thielaviopsis spp., Thyronectria spp., Trachysphaera spp., Uromyces spp., Ustilago spp., Venturia spp., Verticillium spp. and combinations thereof. In a further embodiment, the at least one pathogen comprises Botrytis cinerea.

In another embodiment, the at least one pathogen is selected from the group consisting of Erwinia spp., Pantoea spp., Pectobacterium spp., Pseudomonas spp., Ralstonia spp., Xanthomonas spp.; Salmonella spp., Escherichia spp., Lactobacillus spp., Leuconostoc spp., Listeria spp., Shigella spp., Staphylococcus spp., Candida spp., Debaryomyces spp., Bacillus spp., Campylobacter spp., Clavibacter spp., Clostridium spp., Cryptosporidium spp., Giardia spp., Vibrio spp., Yersinia spp. and combinations thereof.

In another embodiment, the plants or plant parts comprise transgenic plants or transgenic plant parts. In another embodiment, the plants or plant parts are selected from the group consisting of corn, wheat, cotton, rice, soybean, and canola. In another embodiment, the plants or plant parts are selected from the group consisting of fruit, vegetables, nursery, turf and ornamental crops. In a further embodiment, the fruit is selected from the group consisting of banana, pineapple, citrus including oranges, lemon, lime, grapefruit, and other citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries including strawberry, blueberry, raspberry, blackberry, cranberry, currants and other types of berries. In a further embodiment, the vegetable is selected from the group consisting of tomato, potato, sweet potato, cassava, pepper, bell pepper, carrot, celery, squash, eggplant, cabbage, cauliflower, broccoli, asparagus, mushroom, onion, garlic, leek, and snap bean. A further embodiment, the flower or flower part is selected from the group consisting of roses, carnations, orchids, geraniums, lily or other ornamental flowers. A further embodiment, the meat is selected from the group of beef, bison, chicken, deer, goat, turkey, pork, sheep, fish, shellfish, mollusks, or dry-cured meat products.

In another embodiment, the plants or plant parts are selected from the group consisting of banana, pineapple, citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries. In a further embodiment, the plants or plant parts comprise berry or berries. In another further embodiment, the berries are selected from the group consisting of strawberry, blueberry, raspberry, blackberry, cranberry, and combinations thereof. In a further embodiment, citrus is selected from the group consisting of orange, lemon, lime, and grapefruit.

In one embodiment, the at least one target sequence is selected from SEQ ID NOs: 1-13. In another embodiment, the plurality of oligonucleotide primers comprises at least one sequence selected from SEQ ID NOs: 14-29, In another embodiment, the plurality of oligonucleotide primers comprises at least one sequence selected from SEQ ID NOs: 30-45. In another embodiment, the plurality of oligonucleotide primers comprises at least one sequence selected from SEQ ID NOs: 46-61.

In another aspect, provided are methods for detecting at least one pathogen affecting meats, plants, or plant parts. The methods comprise
(a) providing a sample of the meats, plants, or plant parts;
(b) performing a nucleic acid based amplification from the sample using a plurality of oligonucleotide primers for at least one target sequence; and
(c) determining risk level of the at least one pathogen from the sample based on a multi-tier risk system.

In one embodiment of the methods provided, the multi-tier risk system comprises three tiers including low risk, medium risk, and high risk. In another embodiment, the nucleic acid based amplification comprises quantitative polymerase chain reaction (qPCR) or recombinase polymerase amplification (RPA). In another embodiment, the nucleic acid based amplification comprises isothermal nucleic acid amplification. In a further embodiment, the nucleic acid based amplification comprises recombinase polymerase amplification (RPA).

In another embodiment, the at least one pathogen is selected from the group consisting of Acremonium spp., Albugo spp., Alternaria spp., Ascochyta spp., Aspergillus spp., Botryodiplodia spp., Botryospheria spp., Botrytis spp., Byssochlamys spp., Candida spp., Cephalosporium spp., Ceratocystis spp., Cercospora spp., Chalara spp., Cladosporium spp., Colletotrichum spp., Cryptosporiopsis spp., Cylindrocarpon spp., Debaryomyces spp., Diaporthe spp., Didymella spp., Diplodia spp., Dothiorella spp., Elsinoe spp., Fusarium spp., Geotrichum spp., Gloeosporium spp., Glomerella spp., Helminthosporium spp., Khuskia spp., Lasiodiplodia spp., Macrophoma spp., Macrophomina spp., Microdochium spp., Monilinia spp., Monilochaethes spp., Mucor spp., Mycocentrospora spp., Mycosphaerella spp., Nectria spp., Neofabraea spp., Nigrospora spp., Penicillium spp., Peronophythora spp., Peronospora spp., Pestalotiopsis spp., Pezicula spp., Phacidiopycnis spp., Phoma spp., Phomopsis spp., Phyllosticta spp., Phytophthora spp., Polyscytalum spp., Pseudocercospora spp., Pyricularia spp., Pythium spp., Rhizoctonia spp., Rhizopus spp., Sclerotium spp., Sclerotinia spp., Septoria spp., Sphaceloma spp., Sphaeropsis spp., Stemphyllium spp., Stilbella spp., Thielaviopsis spp., Thyronectria spp., Trachysphaera spp., Uromyces spp., Ustilago spp., Venturia spp., Verticillium spp. and combinations thereof. In a further embodiment, the at least one pathogen comprises Botrytis cinerea.

In another embodiment, the at least one pathogen is selected from the group consisting of Erwinia spp., Pantoea spp., Pectobacterium spp., Pseudomonas spp., Ralstonia spp., Xanthomonas spp.; Salmonella spp., Escherichia spp., Lactobacillus spp., Leuconostoc spp., Listeria spp., Shigella spp., Staphylococcus spp., Candida spp., Debaryomyces spp., Bacillus spp., Campylobacter spp., Clavibacter spp., Clostridium spp., Cryptosporidium spp., Giardia spp., Vibrio spp., Yersinia spp. and combinations thereof.

In another embodiment, the plants or plant parts comprise transgenic plants or transgenic plant parts. In another embodiment, the plants or plant parts are selected from the group consisting of corn, wheat, cotton, rice, soybean, and canola. In another embodiment, the plants or plant parts are selected from the group consisting of fruit, vegetables, nursery, turf and ornamental crops. In a further embodiment, the fruit is selected from the group consisting of banana, pineapple, citrus including oranges, lemon, lime, grapefruit, and other citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries including strawberry, blueberry, raspberry, blackberry, cranberry, currants and other types of berries. In a further embodiment, the vegetable is selected from the group consisting of tomato, potato, sweet potato, cassava, pepper, bell pepper, carrot, celery, squash, eggplant, cabbage, cauliflower, broccoli, asparagus, mushroom, onion, garlic, leek, and snap bean. A further embodiment, the flower or flower part is selected from the group consisting of roses, carnations, orchids, geraniums, lily or other ornamental flowers. A further embodiment, the meat is selected from the group of beef, bison, chicken, deer, goat, turkey, pork, sheep, fish, shellfish, mollusks, or dry-cured meat products.

In another embodiment, the plants or plant parts are selected from the group consisting of banana, pineapple, citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries. In a further embodiment, the plants or plant parts comprise berry or berries. In another further embodiment, the berries are selected from the group consisting of strawberry, blueberry, raspberry, blackberry, cranberry, and combinations thereof. In a further embodiment, citrus is selected from the group consisting of orange, lemon, lime, and grapefruit.

In one embodiment, the at least one target sequence is selected from SEQ ID NOs: 1-13. In another embodiment, the plurality of oligonucleotide primers comprises at least one sequence selected from SEQ ID NOs: 14-29. another embodiment, the plurality of oligonucleotide primers comprises at least one sequence selected from SEQ ID NOs: 30-45. In another embodiment, the plurality of oligonucleotide primers comprises at least one sequence selected from SEQ ID NOs: 46-61.

In another aspect, provided are methods for detecting at least one pathogen affecting meats, plants, or plant parts. The methods comprise
(a) providing a sample of the meats, plants, or plant parts;
(b) performing a nucleic acid based amplification from the sample using a plurality of oligonucleotide primers for at least one target sequence; and
(c) determining number of spores of the at least one pathogen in the sample.

In one embodiment of the methods provided, the number of spores in the sample is from 10 to 10,000 spores; from 25 to 5,000 spores; from 50 to 2,500 spores; or from 100 to 1,000 spores of the at least one pathogen. In another embodiment, the nucleic acid based amplification comprises quantitative polymerase chain reaction (qPCR) or recombinase polymerase amplification (RPA). In another embodiment, the nucleic acid based amplification comprises isothermal nucleic acid amplification. In a further embodiment, the nucleic acid based amplification comprises recombinase polymerase amplification (RPA).

In another embodiment, the at least one pathogen is selected from the group consisting of Acremonium spp., Albugo spp., Alternaria spp., Ascochyta spp., Aspergillus spp., Botryodiplodia spp., Botryospheria spp., Botrytis spp., Byssochlamys spp., Candida spp., Cephalosporium spp., Ceratocystis spp., Cercospora spp., Chalara spp., Cladosporium spp., Colletotrichum spp., Cryptosporiopsis spp., Cylindrocarpon spp., Debaryomyces spp., Diaporthe spp., Didymella spp., Diplodia spp., Dothiorella spp., Elsinoe spp., Fusarium spp., Geotrichum spp., Gloeosporium spp., Glomerella spp., Helminthosporium spp., Khuskia spp., Lasiodiplodia spp., Macrophoma spp., Macrophomina spp., Microdochium spp., Monilinia spp., Monilochaethes spp., Mucor spp., Mycocentrospora spp., Mycosphaerella spp., Nectria spp., Neofabraea spp., Nigrospora spp., Penicillium spp., Peronophythora spp., Peronospora spp., Pestalotiopsis spp., Pezicula spp., Phacidiopycnis spp., Phoma spp., Phomopsis spp., Phyllosticta spp., Phytophthora spp., Polyscytalum spp., Pseudocercospora spp., Pyricularia spp., Pythium spp., Rhizoctonia spp., Rhizopus spp., Sclerotium spp., Sclerotinia spp., Septoria spp., Sphaceloma spp., Sphaeropsis spp., Stemphyllium spp., Stilbella spp., Thielaviopsis spp., Thyronectria spp., Trachysphaera spp., Uromyces spp., Ustilago spp., Venturia spp., Verticillium spp. and combinations thereof. In a further embodiment, the at least one pathogen comprises Botrytis cinerea.

In another embodiment, the at least one pathogen is selected from the group consisting of Erwinia spp., Pantoea spp., Pectobacterium spp., Pseudomonas spp., Ralstonia spp., Xanthomonas spp.; Salmonella spp., Escherichia spp., Lactobacillus spp., Leuconostoc spp., Listeria spp., Shigella spp., Staphylococcus spp., Candida spp., Debaryomyces spp., Bacillus spp., Campylobacter spp., Clavibacter spp., Clostridium spp., Cryptosporidium spp., Giardia spp., Vibrio spp., Yersinia spp. and combinations thereof.

In another embodiment, the plants or plant parts comprise transgenic plants or transgenic plant parts. In another embodiment, the plants or plant parts are selected from the group consisting of corn, wheat, cotton, rice, soybean, and canola. In another embodiment, the plants or plant parts are selected from the group consisting of fruit, vegetables, nursery, turf and ornamental crops. In a further embodiment, the fruit is selected from the group consisting of banana, pineapple, citrus including oranges, lemon, lime, grapefruit, and other citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries including strawberry, blueberry, raspberry, blackberry, cranberry, currants and other types of berries. In a further embodiment, the vegetable is selected from the group consisting of tomato, potato, sweet potato, cassava, pepper, bell pepper, carrot, celery, squash, eggplant, cabbage, cauliflower, broccoli, asparagus, mushroom, onion, garlic, leek, and snap bean. A further embodiment, the flower or flower part is selected from the group consisting of roses, carnations, orchids, geraniums, lily or other ornamental flowers. A further embodiment, the meat is selected from the group of beef, bison, chicken, deer, goat, turkey, pork, sheep, fish, shellfish, mollusks, or dry-cured meat products.

In another embodiment, the plants or plant parts are selected from the group consisting of banana, pineapple, citrus, grapes, watermelon, cantaloupe, muskmelon, and other melons, apple, peach, pear, cherry, kiwifruit, mango, nectarine, guava, papaya, persimmon, plum, pomegranate, avocado, fig, and berries. In a further embodiment, the plants or plant parts comprise berry or berries. In another further embodiment, the berries are selected from the group consisting of strawberry, blueberry, raspberry, blackberry, cranberry, and combinations thereof. In a further embodiment, citrus is selected from the group consisting of orange, lemon, lime, and grapefruit.

In one embodiment, the at least one target sequence is selected from SEQ ID NOs: 1-13. In another embodiment, the plurality of oligonucleotide primers comprises at least one sequence selected from SEQ ID NOs: 14-29. another embodiment, the plurality of oligonucleotide primers comprises at least one sequence selected from SEQ ID NOs: 30-45. In another embodiment, the plurality of oligonucleotide primers comprises at least one sequence selected from SEQ ID NOs: 46-61.

In another embodiment, provided are diagnostic kits for detecting at least one pathogen affecting meats, plants, or plant parts. The diagnostic kits comprise a plurality of oligonucleotide primers comprises at least one sequence selected from SEQ ID NOs: 14-29.

In another embodiment, provided are diagnostic kits for detecting at least one pathogen affecting meats, plants, or plant parts. The diagnostic kits comprise a plurality of oligonucleotide primers comprises at least one sequence selected from SEQ ID NOs: 30-45.

In another embodiment, provided are diagnostic kits for detecting at least one pathogen affecting meats, plants, or plant parts. The diagnostic kits comprise a plurality of oligonucleotide primers comprises at least one sequence selected from SEQ ID NOs: 46-61. In another aspect, provided are combinations of oligonucleotide primers for detecting at least one pathogen affecting meats, plants, or plant parts, wherein the primers have different sensitivity for detecting at least one target sequences. In one embodiment, the at least one target sequence is selected from SEQ ID NOs: 1-13. In another embodiment, the oligonucleotide primers comprise at least one sequence selected from SEQ ID NOs: 14-29. In another embodiment, the oligonucleotide primers comprise at least one sequence selected from SEQ ID NOs: 3045. In another embodiment, the oligonucleotide primers comprise at least one sequence selected from SEQ ID NOs: 46-61.

In another aspect, provided are methods for sampling calyx from strawberry for detecting at least one pathogen affecting meats, plants, or plant parts. The method comprise

(a) removing the calyx from the strawberry;
(b) homogenizing the removed calyx, and
(c) performing a nucleic acid based amplification from the sample using a plurality of oligonucleotide primers for at least one target sequence.

In one embodiment of the methods provided, the nucleic acid based amplification comprises quantitative polymerase chain reaction (qPCR) or recombinase polymerase amplification (RPA). In another embodiment, the nucleic acid based amplification comprises isothermal nucleic acid amplification. In a further embodiment, the nucleic acid based amplification comprises recombinase polymerase amplification (RPA).

In another embodiment, the at least one pathogen is selected from the group consisting of Acremonium spp., Albugo spp., Alternaria spp., Ascochyta spp., Aspergillus spp., Botryodiplodia spp., Botryospheria spp., Botrytis spp., Byssochlamys spp., Candida spp., Cephalosporium spp., Ceratocystis spp., Cercospora spp., Chalara spp., Cladosporium spp., Colletotrichum spp., Cryptosporiopsis spp., Cylindrocarpon spp., Debaryomyces spp., Diaporthe spp., Didymella spp., Diplodia spp., Dothiorella spp., Elsinoe spp., Fusarium spp., Geotrichum spp., Gloeosporium spp., Glomerella spp., Helminthosporium spp., Khuskia spp., Lasiodiplodia spp., Macrophoma spp., Macrophomina spp., Microdochium spp., Monilinia spp., Monilochaethes spp., Mucor spp., Mycocentrospora spp., Mycosphaerella spp., Nectria spp., Neofabraea spp., Nigrospora spp., Penicillium spp., Peronophythora spp., Peronospora spp., Pestalotiopsis spp., Pezicula spp., Phacidiopycnis spp., Phoma spp., Phomopsis spp., Phyllosticta spp., Phytophthora spp., Polyscytalum spp., Pseudocercospora spp., Pyricularia spp., Pythium spp., Rhizoctonia spp., Rhizopus spp., Sclerotium spp., Sclerotinia spp., Septoria spp., Sphaceloma spp., Sphaeropsis spp., Stemphyllium spp., Stilbella spp., Thielaviopsis spp., Thyronectria spp., Trachysphaera spp., Uromyces spp., Ustilago spp., Venturia spp., Verticillium spp. and combinations thereof. In a further embodiment, the at least one pathogen comprises Botrytis cinerea.

In another embodiment, the at least one pathogen is selected from the group consisting of Erwinia spp., Pantoea spp., Pectobacterium spp., Pseudomonas spp., Ralstonia spp., Xanthomonas spp.; Salmonella spp., Escherichia spp., Lactobacillus spp., Leuconostoc spp., Listeria spp., Shigella spp., Staphylococcus spp., Candida spp., Debaryomyces spp., Bacillus spp., Campylobacter spp., Clavibacter spp., Clostridium spp., Cryptosporidium spp., Giardia spp., Vibrio spp., Yersinia spp. and combinations thereof.

In one embodiment, the at least one target sequence is selected from SEQ ID NOs: 1-13. In another embodiment, the plurality of oligonucleotide primers comprises at least one sequence selected from SEQ ID NOs: 14-29. In another embodiment, the plurality of oligonucleotide primers comprises at least one sequence selected from SEQ ID NOs: 30-45. 111 another embodiment, the plurality of oligonucleotide primers comprises at least one sequence selected from SEQ ID NOs: 46-61,

Those skilled in the art would understand certain variation can exist based on the disclosure provided. Thus, the following examples are given for the purpose of illustrating the invention and shall not be construed as being a limitation on the scope of the invention or claims.

### EXAMPLES

### Example 1

### Identification of Advantaged Gene Targets for Detection of Botrytis cinerea

The published Botrytis cinerea (BC) genomes (T.4 and B05.10) are computationally analyzed to determine the highest copy number regions to facilitate the development of a sensitive DNA based diagnostic (see Table 1), where the ribosomal IGS, tubulin, and cutinase genes have been analyzed in multiple academic publications. The highest copy number targets contained ˜40 copies per genome. One of the targets, BC Target 3 is identified as encoding a 5S ribosomal RNA. The sequences of each gene target are listed as SEQ ID NOs: 1-13. Quantitative real-time PCR (qPCR) assays are developed to validate the computational predictions.

TABLE 1
High copy number targets identified by computational analysis

| Target | T.4 | B05.10 |
|---|---|---|
| BC Target 1 | 41 | 45 |
| BC Target 3 | 38 | 40 |
| BC Target 4 | 20 | 21 |
| BC Target 5 | 4 | 20 |
| BC Target 6 | 5 | 24 |
| BC Target 7 | 25 | 20 |
| BC Target 8 | 8 | 18 |
| BC Target 9 | 13 | 18 |
| BC Target 10 | 26 | 27 |
| BC Target 11 | 8 | 14 |
| Ribosomal IGS | 1 | 5 |
| Tubulin | 1 | 1 |
| Cutinase | 1 | 1 |

Primers for qPCR are listed in Table 2, where a dsDNA binding fluorescent probe, EvaGreen dye, is used for qPCR. EvaGreen dye is a superior version of the SYBR green dye and can be used in SYBR green assays. Botrytis gDNA is isolated using a Qiagen Plant DNeasy kit.

For each qPCR reaction the following reagents are mixed together:
1.8 μL Forward Primer (50 μM)
1.8 μL Reverse Primer (50 μM)
5.0 μL 20× EvaGreen dye
50 μL TaqMan Fast Master-mix no amperase (2×)
31.4 μL H₂O

The Botrytis gDNA (10 ng/μL) s sequentially 10 fold serially diluted to a concentration of 1 pg/μL (24 copies/μL). For each reaction, 2 μL of appropriately diluted template gDNA is added to the well followed by 18 μL of the reaction mix prepared above. The plate is then spun down for 5 minutes at 2200 RCF. The amplification reaction is performed on an Applied Biosystems StepOne Plus real time PCR system (Foster City, Calif.). Cycling conditions are 95° C. for 20 seconds for denaturation, 40 cycles of 95° C. for 3 seconds and 60° C. for 30 seconds.

TABLE 2
qPCR primers for selected Botrytis gene targets

| SEQ | | | |
|---|---|---|---|
| ID NO. | Sequence Name | Sequence | nmoles |
| 14 | BC_target1_F1 | CCT AAG CGA ATG CGA | 34.3 |
| | | AAG AG | |
| 15 | BC_target1_R1 | CGA GAA GGA TAC GGA | 32.2 |
| | | AGA CG | |
| 16 | BC_target7_F1 | CAG GCT GTA GAA TCA | 25 |
| | | CCA ACG | |
| 17 | BC_target7_R1 | CTA AGG CTT TCC TTG | 39.2 |
| | | GAT GC | |
| 18 | BC_target3_F1 | CTG AAG AGA ATT GGG | 24.3 |
| | | CAT CC | |
| 19 | BC_target3_R1 | CAT ACA ACA GTG GGG | 30.3 |
| | | ATT CG | |
| 20 | BC_target4_F1 | CAC CAT GGG GAT GGT | 30.7 |
| | | GAA T | |
| 21 | BC_target4_R1 | TTC GGC ACT ACA GCA | 31.8 |
| | | ATA CG | |
| 22 | BC_target5_F1 | CCC TCT TTT GGA CCA | 37.4 |
| | | CCT AA | |
| 23 | BC_target5_R1 | CTG GTG ATC GGG AAA | 39 |
| | | TTG AG | |
| 24 | BC_target6_F1 | AAG CAC TAC CTC CCA | 25 |
| | | ACT TCA | |
| 25 | BC_target6_R1 | GCA ATT GCA AAA AGT | 32 |
| | | GCT G | |
| 26 | BC_target8_F1 | CTA CTA GCG TGC CCT | 38.7 |
| | | GCT TC | |
| 27 | BC_target8_R1 | AAG GCA CGG GTA AAG | 27.8 |
| | | ACG TA | |
| 28 | BC_target9_F1 | CAT AGA GCA AGT GGC | 22 |
| | | TAC ACG | |
| 29 | BC_target9_R1 | TTG AGT GCC CAG CTC | 41.1 |
| | | TTA CC | |

The C_{T} values can be converted to copies per genome assuming the tubulin gene is present as a single copy. This assumption is strongly supported by bioinformatic knowledge of the genome of Botrytis and other related fungi. The copies per genome are calculated using the following equation:

(1+PCR_{efficiency})^{(Ct}^{_{tubulin}}^{-Ct}^{_{Target}}⁾

Based on the experimental results shown in Table 3, the targets are ranked in the following order: Ribosomal IGS>(BC Target 1 and BC Target 3)>(BC Target 7 and BC Target 8)>(BC Target 5, BC Target 6, and BC Target 9)>Tubulin>BC Target 4.

The ribosomal IGS, BC Target 1 and BC Target 3 appear to be genes of high copy numbers. BC Target 7 and BC Target 8 appear at 2-3 folds lower copies per genomes. BC Target 5, BC Target 6, and BC Target 9 performed worse in these experiments than other selected targets in the initial qPCR experiment. BC Target 1 is therefore selected for further analysis.

TABLE 3
The calculated copies per genome for each target tested

| Target | Experimental Copy Number | Length (bp) |
|---|---|---|
| BC Target 1 | 26.1 ± 2.3 | 245 |
| BC Target 3 | 31.9 ± 3.7 | 133 |
| BC Target 4 | 0.5 ± 0 | 194 |
| BC Target 5 | 2.1 ± 0.1 | 478 |
| BC Target 6 | 3.9 ± 0.8 | 312 |
| BC Target 7 | 10.0 ± 1.2 | 139 |
| BC Target 8 | 12.8 ± 1.4 | 161 |
| BC Target 9 | 3.1 ± 0.3 | 240 |
| BC Tubulin | 1.0 | 928 |
| BC IGS p1 | 51.3 ± 2.3 | 300 |
| BC IGS p2 | 56.6 ± 6.9 | 300 |

### Example 2

### Design and Evaluation of RPA Primer Sets for Amplification of Botrytis cinerea Target 1

BC Target 1 from Example 1 is selected for development of a primer set for use in recombinase polymerase amplification (RPA). There are ˜25 copies of BC Target 1 per Botrytis genome and the sequence has favorable GC content (%40) for RPA. The BC Target 1 genetic element is 245 bases, which gives some room to screen a larger number of primers relative to BC Target 3.

No known computer software or models exist for development of primer sets for RPA. To develop a primer set, a relatively large screening effort must be performed for each target. A multiple sequence alignment of the BC Target 1 sequences in the Botrytis genome is performed to determine the best region for RPA amplification. The BC Target 1 genetic element is present in many similar, but non-identical copies in the genome. It is provided to design primers for the most conserved regions of BC Target 1. Accordingly, eight forward (F1-F8) and eight reverse primers (R1-R8) (SEQ ID NOs: 30-45) are selected for the most conserved regions of the BC Target 1 genetic element.

Primers are then screened using RPA, with the exception of the addition of lx EvaGreen dye. The amplification reaction is performed on an Applied Biosystems StepOne Plus real time PCR system. Amplification is monitored by an increase in fluorescence due to binding of the EvaGreen dye to double stranded DNA produced by the RPA reaction.

The increase in fluorescence in the absence of target DNA, prompted the analysis of melting curves to determine if a desired amplicon is produced. Melt curve analysis is performed on each primer pair in the presence and absence of target DNA to determine a target DNA dependent effect on the melt curve. It is provided that if specific amplification is taking place, then in the presence of target DNA there would be a sharp single peak in the melt curve.

The majority of screened primer pairs showed no difference in melt curve in the presence or absence of target DNA. The R2F3 primer pair for BC Target 1 shows the best overall performance in the initial screen. The reaction products are then analyzed on the BioAnalyzer. Multiple reaction products can be identified in the BioAnalyzer under certain circumstances.

### Example 3

### Design and Evaluation of RPA Primer Sets for Amplification of Botrytis cinerea Ribosomal Intergenic Spacer

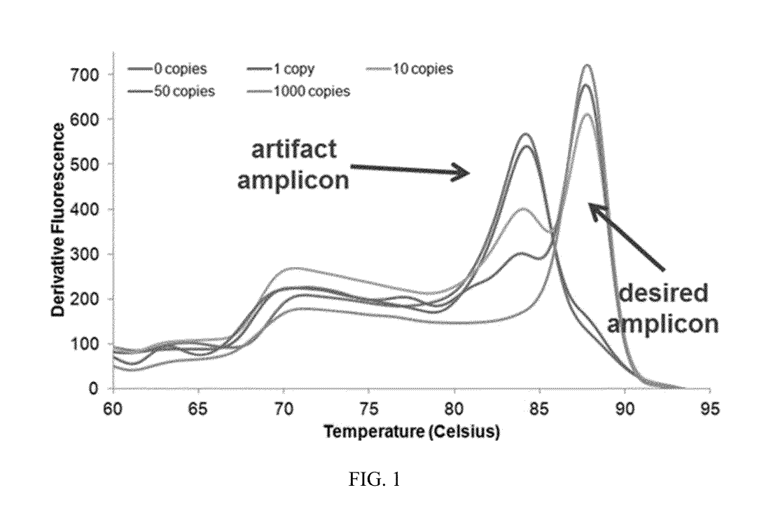

The selected BC Target 1 can produce multiple amplification products under certain circumstance. Because the ribosomal IGS target performed best in the qPCR assays and there is precedent in the literature that this is a sensitive target for the detection of Botrytis, the Ribosomal Intergenic Spacer (IGS) is also selected for further development. Primers are designed and set forth in SEQ ID NOs: 46-61. The primers are screened initially with the EvaGreen dye and melt curve analysis strategy. The initial screen results are shown in FIG. 1. For this particular screen, about 250 copies of Botrytis cinerea genomic DNA is used per reaction.

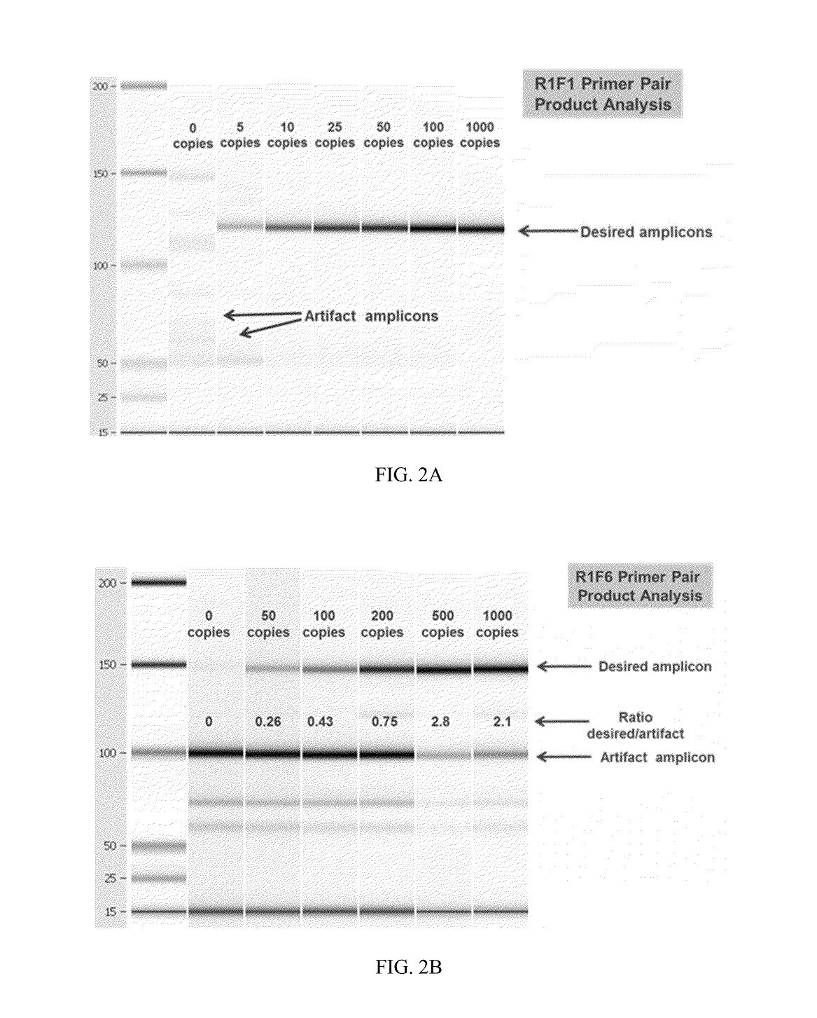

The R1F1 and R1F3 primer pairs show strong amplification and good sensitivity. The products of the R1F1 and R1F3 primer pairs are analyzed on the BioAnalyzer. The RPA reactions using these primer pairs show the production of a single amplicon at the expected size in the electropherogram. The specificity and sensitivity of the R1F1 primer pair is further characterized by serial dilutions and analysis of reaction products on the BioAnalyzer. A lower sensitivity primer pair, R1F6, is also further characterized similarly (see FIGS. 2A and 2B)

The R1F1 primer pair shows significantly better sensitivity than the R1F6 primer pair. The R1F1 primer pair shows significant amplification of the desired amplicon at 5 genomic copies. The R1F6 primer pair shows comparable amplification only at 100 genomic copies or higher.

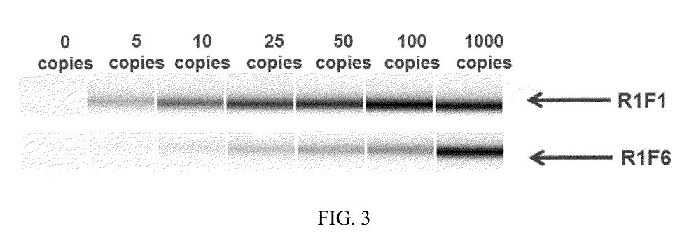

These experiment results demonstrate that primer pairs with differing sensitivities for the same target can be produced. An shown in FIG. 3, the primer pair R1F1 can be useful for detection of Botrytis genomic DNA at a concentrations as low as 5-10 copies, while the R1F6 primer pair can be useful for detection of greater than 100 copies.

### Example 4

### In Vivo Experiment for Detection of Botrytis cinerea on Strawberry Calyx

Calyx of strawberry is selected for in vivo experiment for detection of Botrytis cinerea. Accordingly, calyx is manually removed and then homogenized inside of a plastic bag by grinding or scraping. Prior to homogenization the calyx sample can be spiked with Botrytis spores, Botrytis genomic DNA, or water. For some samples, approximately 5-10 milligrams of Botrytis spores are added to the calyx before homogenization. After homogenization in the plastic bag, one microliter of the calyx homogenate is transferred to fifty microliters of RPA master-mix [containing at least one primer pair (for example R1F1) and RPA basic buffer]. The reaction is incubated at thirty-nine degrees Celsius for twenty minutes and then the products are analyzed on the BioAnalyzer. The calyx homogenate appears a green and very viscous material.

The BioAnalyzer results show a positive signal for samples spiked with Botrytis spores or Botrytis genomic DNA. The negative control reaction (which contained only RPA mix without any calyx added) shows no sign of amplification. Some of the calyx samples that are not spiked with Botrytis show the desired amplicon, suggesting these samples are already infected with Botrytis. Calyx #2 closely matched the negative control, suggesting the strawberry was not infected. The positive control containing only Botrytis spores and no calyx show the strong amplification of the desired amplicon.

Sequence Listing

SEQ ID NO: 1 BC_target1_41-45_hits_per_genome
AGGAAAGGATAGTGTGTGAACGGAGTGAATAACTTCAATTCAATTACCACTGTA
ATATAGCAACTATAATAAAGCCCTAAGCGAATGCGAAAGAGAGTAGCTCTTTCT
GTAAGCCTTTATAAGGCTTACTACTTTCGATACGTAGCTAGCTCTTTAGACAGAA
TACAATTAGACATACAGGACCTACGATATTCGTGGGTGCTACGTCTTCCGTATCC
TTCTCGTACCAACAGATAGTGAGGTTG
SEQ ID NO: 2 BC_target7_20-25_hits_per_genome
TGTTACGACGGATTAGTAACAGGCTGTAGAATCACCAACGTATAGGCTATAATG
GTATTATAGGCCTCAGTGATTCAGCTGCAGTATACCGGGGGATACTAGGCATCCA
AGGAAAGCCTTAGGTATATATATAGTATTAATTATAGAATATTCTAAAAGTATAG
GATACAGTTTTTAGA
SEQ ID NO: 3 BC_target3_38-40_hits_per_genome_5S_ribosomal_RNA
TCTGACACATACGACCATAGACTGAAGAGAATTGGGCATCCCGTCCGCTCTGCCA
TACACAAGCTTCAGATCGGTGGATTAGTAGTTGGGTGGGTGACCACCAGCGAAT
CCCCACTGTTGTATGTTTCTTTTC
SEQ ID NO: 4 BC_target4_20-21_hits_per_genome
AATTTAGAACTGTTGGTTTCACCATGGGGATGGTGAATTCAATATAGTACTATGG
TTCACACTGTTGTAATATTGCTTAAGGTTCTAAAAGCTAAGACTACGAAACGTAT
TGCTGTAGTGCCGAAAGGCGCTAGCACAAGCGCTAGCACGGTCACATGATCACT
ATCCCGACAAGAACCATCACTGTCCTCACA
SEQ ID NO: 5 BC_target5_4-20_hits_per_genome
TAAGTTGAGTACCCCACTTTCGGACCACCCCTCTTTTGGACCACCTAAAAATATA
TATATATATGAATTTTAAACTTCAATAACTCAACCACTATTCAACTTCAATACAAT
CCCCTGTAGTGTCAGTTTCATAAATACTATCAGAGTCTTTTATCTCAATTTCCCGA
TCACCAGCTTCAATTTGAGCTTGATATATAGCTTCTATCCCTGCAAACCTCGAATT
TGGACTAGTTTTTACCATCCTTCTTTTTTTAGGTATAATCTCTTCTAATTTTTGCTC
CAATTGCTTTATTCGTTTCTTGGACTGTACAAGTTCATAGTCCTTAGCATCAAATC
CTTTTTGAATCTTTCGAAAAAGCAGCCGGCGAGTTGGAATATCGGTCTCATCAAC
TTTCTCCATTATATCAGCATATTTTCGAATATCACTTCCTTTTTGAGGGGTTTTCCA
TGCAATAAAAGATGAATGTATTTCCTCCAT
SEQ ID NO: 6 BC_target6_5-24_hits_per_genome
TAAGTTGAGTACCCCACTTTCGGACCACCCCTCTTTTGGACCACCTAAAATCTTAC
CCCATTTTAAGCACTACCTCCCAACTTCATCTTTAATAAATCAACAACCACATATT
CAATTGATATAAGATTTTAATATATTATCAATTAAGCTATAACAAAGCCTTATAC
TGAAGATAATATTGCTGCAGCACTTTTTGCAATTGCAGAAGGCATGTCTATATAT
AAGGCTTACTCAGAATATGATATTCCCCACACCACTTTATACAACTATATAAATA
GCCACCTTTCACATAAAAAAGATACACAAAACCTATAGAAGATAGCTCCTATAT
AGGAGAGAGCTTTAGCAAATTGGATTTTAATACAGAAAGCCCTAAAAACTAGCC
CTATCTATTATCAAATACAAAAATTAGGAAAGTCCATTCTCAACCTCGAAAGAGA
TGATTTATTTTTGAACAAGCGATGGATATATAGTTTTTTGAAAAGAAACCTAGAA
ATTAAAACTAAAAGGCAATATAAAATCAATAATGCCTATATCAATAATACAATT
ACCAAAATTATAAGCAAGTTCTTTGAAAAATTAGATTTACTAAC
SEQ ID NO: 7 BC_target8_8-18_hits_per_genome
AGGGTTGGCTTGTGTCACGGCGCCAACTACATGTTCTGTAGTTGCCTTCGTGCCTT
AGGCACGGACTACTAGCGTGCCCTGCTTCCTATAAGTAGGGCCTCACTCTTCCAT
AGCTCTTCCACCCTTATGGTACAATATACGTCTTTACCCGTGCCTTGACAGTTTGT
ACCATCTT
SEQ ID NO: 8 BC_target9_13-18_hits_per_genome
TAATAATTTAATCTTCTTATTGTAAAAGAGTAGAAGGTGGTAATGGTCACACAAG
AAAAGCCTTCGCATATATCAAGCATAGAGCAAGTGGCTACACGTAGTAAAATGG
GGTGAATCACTATATTGCGATAGCGAGGTGAGGGAGGCGGTAAGAGCTGGGCAC
TCAATTCTTCAGGAGACAACTTTAGAAGGTAGAAAATTCGATGATATTTTTAGGT
CTACAGAAGTGAAGCTATAAATACTAAATGTTGATAACACGTGATCCCAGAGTC
ACGTGTTTTCACTCTCACATTATCGATTGGAA
SEQ ID NO: 9 BC_target10_26-27_hits_per_genome
ATCACTCACTTACTTCACTTTCAATTTATTCTTCAATCAGAAGCTTTACCACTATA
CCATGCCATACAGATTGTACTATTTATATACATTATCTACCTAGCTTTGATTTATA
TATTCATATTCAATTCAATTACTAATCGAATTCAATATAAATAAAGTATTCATCAT
CTTAAACTAGAATTCAAGAATTTCACTAAGTCCCTTTGGAATAAAAATTTCTAAT
CATTTACAAAAGAAAAAGCCCCCTAATCAATACTTTAAAAACGCTTTTTTCAATA
CATTATAAAAATATTGAATATTTTCTGGGGCTGATAAAGCGGCCACTTCGTCAAT
CGCGGATTCTGCCACCACAGGGGGTATATATACTAC
SEQ ID NO: 10 BC_target11_8-14_hits_per_genome
GGTTTATCAGATCAGTGAGTGGGTCATATCAGTGAATGGGTCATTTGAAAATACA
TCAAAATTAGGCCTTTTTCAATATTCATATTTTAATAGCTAATCATTATTTTGAAA
AAAGAAAAGAATTTTCAATAACAATAAGAAATTAGCTTCTTATAAATTTAGTTTT
TTTTAT
SEQ ID NO: 11 BC_IGS_1
TGGTTCGACTGTAGTCCCTAGGAACGCCCTCTGAGTGTCCTAGGAATGCCCCCGG
TGAGCCCTTGGTCTAAAGCCGTATAGGTGACTAGTTAACCCCATATAGTTTGTGC
GAGTACACACACTACTACCGGTGAGCAGGCTGTAATTTCAATGTGCAGAATCTGT
CCCCGGTGAGCGCAGGTCACCTTGCAATGAGTGGACAGCATGTTTGAAATGCGA
TTAATTGTTGCTCCCGGTGAGCCCACTAAATAATTCTGGGAGTTGGCCATCTCAT
ATTTCATCCCCGGTGAGCCCAAGATA
SEQ ID NO: 12 BC_tubulin_gene
ACATCAGATATCTATTCCTCGCCCTCAATTGGGACCTCCTCTTCGTACTCCTCCTC
TCCCTCAGAGATCGAGGCATCCTGGTATTGTTGATACTCGGAAACCAAATCGTTC
ATGTTGGACTCAGCCTCAGTGAACTCCATCTCGTCCATACCTTCACCAGTGTACC
AATGCAAGAAAGCCTTTCTTCTGAACATAGCAGTGAATTGATCACCGACACGCTT
GAAAAGTTCTTGGATGGATGTCGAGTTACCAACGAAGGTGGAGGACATCTTGAG
ACCACGGGGAGGAATGGAGCAAAGGGCGGTTTGGACGTTGTTAGGGATCCACTC
AACGAAGTAGGATGAGTTCTTGTTTTGGACGTTGCGCATTTGGTCCTCAACCTCC
TTCATGGAAACCTTACCACGGCTACAGAAAGTTAGTTTCTACAAGATTTTGGCAG
ATTGATTACAGGGCAAACTTACAAAATGGCAGAGCATGTCAAGTAACGACCGTT
ACGGAAATCGGAAGCGGCCATCATGTTCTTAGGGTCGTACATTTGTTGAGTCAAC
TCTGGAACGGTGACAGCACGGAAAGAGTGTGCGCCACGACTGGTCAAAGGAGCA
AATCCAACCATGAAGAAATGGAGACGGGGGAATGGAACCATGTTAACAGCCAAC
TTTCGGAGATCTGAGTTAAGTTGACCAGGGAAACGGAGACAGGTGGTAACACCG
GACATGACGGCGGAAACCAAGTGGTTAAGATCTCCGTAAGATGGGTTGCTGAGC
TTCAAGGTTCTCATGCAAATATCGTAAAGAGCCTCGTTATCGATACAGAAGGTCG
CGTCAGAGTTCTCAACCAATTGATGGACAGAGAGAGTTGCGTTATATGGCTCGAC
AACGGTATCGGAAACCTTTGGCGATGGGACGACGGAGAAGGTAGCCATCATACG
SEQ ID NO: 13 BC_Cutinase
AAAAGAATCTCAACTTAAATGGAAATTCATTCTGAGCTGATACTCGTTGCCGTCA
CATAAAATATAAAGTGATTGACATCGAGAAAGTTTCTCAATCTACCTAGTTTGCA
TCGCTTTGAGCAACTCATCACTCCGGCTCGGCAGATGTTAGCTCGAATGAAAGAT
TTGATGGTAGGCTTTCCTGTCGAATTTGCCAGTTGAATTTGCCAGTATGGTGTGA
ATGCGCTGTATGTTCTAGCGACGCCTAATACTAGATGTCTAAGATGTCTAGTAGT
AGCTCGACGCCGTGACATGCCGTCACCATGAAATTTGCTGAGTTTGGTTGTATAA
AGAAGAGGGAAAGGAATGAAAACCAATACACGGAGAGAAATAGTATAAAGATT
GGATTGAATGGAAAGTGTTTACTTCCTCTGCGTTAACTCTAGTTTCCGGATAGTA
CCGCGGGATCTTGCTGGGCAGGCATGAGCTATGTGGAGCTTCAAGCTTTCTCAAT
ATGGGGTAGCCTTATGTCCCTTCCCTTGTCCTTGCTGTCGATCTCACCATTTTCCA
TTTCTCTTCACCTCTTTCTCCTCCGTGATTCAACCACACCTCTTAGAATCTTTAATG
CCTCGGCAGTTGAAGACATACACGGGCCTCGTCAATTATCGCACATTGTACTACT
CACCAACTTAATGAAATACTGGCATCTAAACACGGTATTCAAAAGATGCGAGAT
GTACAGACAGACACTCGCAGGTCATGACAAATTCCCCGTCGGACTTCCACATTGG
AATTTTGAGAGTCCAAGCAAAAAAGTTACAATGGTGTTATGTTGCATCACAATCA
AATCTTCCTTACTTTTTCTCCACACAGCCACCACCATCCTCCTTATGCTTCTTTCAT
CCTTAACGTTTCAAAAAGTCGGATTCATCTGAAAAAGTT
SEQ ID NO: 14 BC_target1_F1
CCTAAGCGAATGCGAAAGAG
SEQ ID NO: 15 BC_target1_R1
CGAGAAGGATACGGAAGACG
SEQ ID NO: 16 BC_target7_F1
CAGGCTGTAGAATCACCAACG
SEQ ID NO: 17 BC_target7_R1
CTAAGGCTTTCCTTGGATGC
SEQ ID NO: 18 BC_target3_F1
CTGAAGAGAATTGGGCATCC
SEQ ID NO: 19 BC_target3_R1
CATACAACAGTGGGGATTCG
SEQ ID NO: 20 BC_target4_F1
CACCATGGGGATGGTGAAT
SEQ ID NO: 21 BC_target4_R1
TTCGGCACTACAGCAATACG
SEQ ID NO: 22 BC_target5_F1
CCCTCTTTTGGACCACCTAA
SEQ ID NO: 23 BC_target5_R1
CTGGTGATCGGGAAATTGAG
SEQ ID NO: 24 BC_target6_F1
AAGCACTACCTCCCAACTTCA
SEQ ID NO: 25 BC_target6_R1
GCAATTGCAAAAAGTGCTG
SEQ ID NO: 26 BC_target8_F1
CTACTAGCGTGCCCTGCTTC
SEQ ID NO: 27 BC_target8_R1
AAGGCACGGGTAAAGACGTA
SEQ ID NO: 28 BC_target9_F1
CATAGAGCAAGTGGCTACACG
SEQ ID NO: 29 BC_target9_R1
TTGAGTGCCCAGCTCTTACC
SEQ ID NO: 30 TARGET1_TDX_1F
AAGCCCTAAGCGAATGCGAAAGAGACTAGCTCTTT
SEQ ID NO: 31 TARGET1_TDX_2F
TARTAAAGCCCTAAGCGAATGCGAAAGAGACTAGC
SEQ ID NO: 32 TARGET1_TDX_3F
WACTGTARTAAAGCCCTAAGCGAATGCGAAAGAGA
SEQ ID NO: 33 TARGET1_TDX_4F
ATAGCWACTGTARTAAAGCCCTAAGCGAATGCGAA
SEQ ID NO: 34 TARGET1_TDX_5F
GTAATATAGCWACTGTARTAAAGCCCTAAGCGAAT
SEQ ID NO: 35 TARGET1_TDX_6F
CCACTGTAATATAGCWACTGTARTAAAGCCCTAAG
SEQ ID NO: 36 TARGET1_TDX_7F
AATTACCACTGTAATATAGCWACTGTARTAAAGCC
SEQ ID NO: 37 TARGET1_TDX_8F
AATTCAATTACCACTGTAATATAGCWACTGTARTAA
SEQ ID NO: 38 TARGET1_TDX_1R
GTTGGTACGAGAAGGATACGGAAGACGTAGCACCC
SEQ ID NO: 39 TARGET1_TDX_2R
TACGAGAAGGATACGGAAGACGTAGCACCCACGAA
SEQ ID NO: 40 TARGET1_TDX_3R
GAAGGATACGGAAGACGTAGCACCCACGAATWKCG
SEQ ID NO: 41 TARGET1_TDX_4R
ATACGGAAGACGTAGCACCCACGAATWKCGTAGGT
SEQ ID NO: 42 TARGET1_TDX_5R
GAAGACGTAGCACCCACGAATWKCGTAGGTCCTGT
SEQ ID NO: 43 TARGET1_TDX_6R
CGTAGCACCCACGAATWKCGTAGGTCCTGTATGTC
SEQ ID NO: 44 TARGET1_TDX_7R
CACCCACGAATWKCGTAGGTCCTGTATGTCTAATT
SEQ ID NO: 45 TARGET1_TDX_8R
ACGAATWKCGTAGGTCCTGTATGTCTAATTGTATT
SEQ ID NO: 46 Ribosomal_IGS_TDx_1F
CGGTGAGCAGGCTGTAATTTCAATGTGCAGAATCT
SEQ ID NO: 47 Ribosomal_IGS_TDx_2F
ACTACCGGTGAGCAGGCTGTAATTTCAATGTGCAG
SEQ ID NO: 48 Ribosomal_IGS_TDx_3F
ACACTACTACCGGTGAGCAGGCTGTAATTTCAATG
SEQ ID NO: 49 Ribosomal_IGS_TDx_4F
TACACACACTACTACCGGTGAGCAGGCTGTAATTT
SEQ ID NO: 50 Ribosomal_IGS_TDx_5F
GCGAGTACACACACTACTACCGGTGAGCAGGCTGT
SEQ ID NO: 51 Ribosomal_IGS_TDx_6F
TTTGTGCGAGTACACACACTACTACCGGTGAGCAG
SEQ ID NO: 52 Ribosomal_IGS_TDx_7F
TATAGTTTGTGCGAGTACACACACTACTACCGGTG
SEQ ID NO: 53 Ribosomal_IGS_TDx_8F
CCCCATATAGTTTGTGCGAGTACACACACTACTAC
SEQ ID NO: 54 Ribosomal_IGS_TDx_1R
GTGGGCTCACCGGGAGCAACAATTAATCGCATTTC
SEQ ID NO: 55 Ribosomal_IGS_TDx_2R
ATTTAGTGGGCTCACCGGGAGCAACAATTAATCGC
SEQ ID NO: 56 Ribosomal_IGS_TDx_3R
GAATTATTTAGTGGGCTCACCGGGAGCAACAATTA
SEQ ID NO: 57 Ribosomal_IGS_TDx_4R
TCCCAGAATTATTTAGTGGGCTCACCGGGAGCAAC
SEQ ID NO: 58 Ribosomal_IGS_TDx_5R
CCAACTCCCAGAATTATTTAGTGGGCTCACCGGGA
SEQ ID NO: 59 Ribosomal_IGS_TDx_6R
GATGGCCAACTCCCAGAATTATTTAGTGGGCTCAC
SEQ ID NO: 60 Ribosomal_IGS_TDx_7R
TATGAGATGGCCAACTCCCAGAATTATTTAGTGGG
SEQ ID NO: 61 Ribosomal_IGS_TDx_8R
TGAAATATGAGATGGCCAACTCCCAGAATTATTTA

We claim:

1. A method of detecting at least one pathogen affecting meats, plants, or plant parts, comprising:
(a) providing a sample of the meats, plants, or plant parts;
(b) performing a nucleic acid based amplification from the sample using a plurality of oligonucleotide primers for at least one target sequence; and
(c) determining presence or absence of the at least one pathogen from the sample.

2. The method of claim 1, wherein the nucleic acid based amplification comprises quantitative polymerase chain reaction (qPCR) or recombinase polymerase amplification (RPA).

3. The method of claim 1, wherein the nucleic acid based amplification comprises recombinase polymerase amplification (RPA).

4. The method of claim 1, wherein the at least one pathogen is selected from the group consisting of Acremonium spp., Albugo spp., Alternaria spp., Ascochyta spp., Aspergillus spp., Botryodiplodia spp., Botryospheria spp., Botrytis spp., Byssochlamys spp., Candida spp., Cephalosporium spp., Ceratocystis spp., Cercospora spp., Chalara spp., Cladosporium spp., Colletotrichum spp., Cryptosporiopsis spp., Cylindrocarpon spp., Debaryomyces spp., Diaporthe spp., Didymella spp., Diplodia spp., Dothiorella spp., Elsinoe spp., Fusarium spp., Geotrichum spp., Gloeosporium spp., Glomerella spp., Helminthosporium spp., Khuskia spp., Lasiodiplodia spp., Macrophoma spp., Macrophomina spp., Microdochium spp., Monilinia spp., Monilochaethes spp., Mucor spp., Mycocentrospora spp., Mycosphaerella spp., Nectria spp., Neofabraea spp., Nigrospora spp., Penicillium spp., Peronophythora spp., Peronospora spp., Pestalotiopsis spp., Pezicula spp., Phacidiopycnis spp., Phoma spp., Phomopsis spp., Phyllosticta spp., Phytophthora spp., Polyscytalum spp., Pseudocercospora spp., Pyricularia spp., Pythium spp., Rhizoctonia spp., Rhizopus spp., Sclerotium spp., Sclerotinia spp., Septoria spp., Sphaceloma spp., Sphaeropsis spp., Stemphyllium spp., Stilbella spp., Thielaviopsis spp., Thyronectria spp., Trachysphaera spp., Uromyces spp., Ustilago spp., Venturia spp., Verticillium spp. and combinations thereof.

5. The method of claim 1, wherein the at least one pathogen is selected from the group consisting of Erwinia spp., Pantoea spp., Pectobacterium spp., Pseudomonas spp., Ralstonia spp., Xanthomonas spp.; Salmonella spp., Escherichia spp., Lactobacillus spp., Leuconostoc spp., Listeria spp., Shigella spp., Staphylococcus spp., Candida spp., Debaryomyces spp., Bacillus spp., Campylobacter spp., Clavibacter spp., Clostridium spp., Cryptosporidium spp., Giardia spp., Vibrio spp., Yersinia spp. and combinations thereof.

6. The method of claim 1, wherein the at least one pathogen comprises Botrytis cinerea.

7. The method of claim 1, wherein the plants or plant parts are selected from the group consisting of banana, pineapple, citrus, grapes, watermelon, cantaloupe, muskmelon, and other melons, apple, peach, pear, cherry, kiwifruit, mango, nectarine, guava, papaya, persimmon, plum, pomegranate, avocado, fig, citrus, and berries.

8. The method of claim 1, wherein the plants or plant parts comprise berry or berries.

9. The method of claim 8, wherein the berries are selected from the group consisting of strawberry, blueberry, raspberry, blackberry, cranberry, and combinations thereof.

10. The method of claim 1, wherein the at least one target sequence is selected from SEQ ID NOs: 1-13.

11. The method of claim 1, wherein the plurality of oligonucleotide primers comprises at least one sequence selected from SEQ ID NOs: 14-29.

12. The method of claim 1, wherein the plurality of oligonucleotide primers comprises at least one sequence selected from SEQ ID NOs: 30-45.

13. The method of claim 1, wherein the plurality of oligonucleotide primers comprises at least one sequence selected from SEQ ID NOs: 46-61.

14. A method of detecting at least one pathogen affecting meats, plants, or plant parts, comprising:
(a) providing a sample of the meats, plants, or plant parts;
(b) performing a nucleic acid based amplification from the sample using a plurality of oligonucleotide primers for at least one target sequence; and
(c) determining risk level of the at least one pathogen from the sample based on a multi-tier risk system.

15. The method of claim 14, wherein the multi-tier risk system comprises three tiers including low risk, medium risk, and high risk.

16. The method of claim 14, wherein the method optionally comprises the step of determining number of spores of the at least one pathogen in the sample.

17. A diagnostic kit for detecting at least one pathogen affecting plants or plant parts, comprising a plurality of oligonucleotide primers comprises at least one sequence selected from SEQ ID NOs: 14-29, or at least one sequence selected from SEQ ID NOs: 30-45, or at least one sequence selected from SEQ ID NOs: 46-61.

18. The method of claim 1, wherein the sample is a strawberry, and wherein the method further comprises the steps of removing a calyx from the strawberry and homogenizing the removed calyx prior to performing the nucleic acid based amplification from the sample using a plurality of oligonucleotide primers for at least one target sequence.

19. The method of claim 18, wherein the nucleic acid based amplification comprises quantitative polymerase chain reaction (qPCR) or recombinase polymerase amplification (RPA).

20. The method of claim 1, wherein the nucleic acid based amplification comprises recombinase polymerase amplification (RPA).
